(19) United States
(12) Patent Application Publication
(10) Pub. No.: US 2025/0380897 A1
(43) Pub. Date: Dec. 18, 2025

(54) METHOD AND SYSTEM FOR ESTIMATING DYNAMIC SEIZURE LIKELIHOOD

(71) Applicant: Neuronostics Limited, Bristol (GB)

(72) Inventors: Wessel Woldman, Bristol (GB); John Robert Terry, Bristol (GB)

(21) Appl. No.: 18/879,961

(22) PCT Filed: Jul. 3, 2023
(86) PCT No.: PCT/EP2023/068247

§ 371 Date: Dec. 30, 2024

(30) Foreign Application Priority Data
Jul. 1, 2022 (GB) 2209730.7

(51) Int. Cl. A61B 5/00; A61B 5/145; A61B 5/291; A61B 5/372

(52) U.S. Cl. CPC A61B 5/4094; A61B 5/14532; A61B 5/291; A61B 5/372; A61B 5/4806; A61B 5/7264

(57) ABSTRACT

There is presented a system for estimating the change over time of a likelihood of future epileptiform activity of a patient. A memory stores a model for estimating the likelihood of epileptiform activity of the patient. The model is configured to use data representing coupled brain activity for a plurality of different brain regions of the patient. The coupled brain activity associated with a brain network. The processor is configured to determine one or more parameters for the model using one or more measurements of at least one physiological factor of the patient. The processor is configured to fit the model, using the one or more parameters, to at least a first value associated with likelihood of epileptiform activity derived from one or more measurements of the patient's brain activity; the one or more measurements of the patient's brain activity for determining the data representing coupled brain activity in the model. The processor is configured to estimate the change over time of the likelihood of future epileptiform activity from the fitted model.

### FIELD OF THE INVENTION

In one aspect, the present invention is in the field of electroencephalogram (EEG), in particular the cap or headset for providing EEG readings. In another aspect the present invention is in the field of methods and systems for detecting and monitoring epilepsy and estimating changes to seizure likelihood over time. Such a system may utilise wearables.

### BACKGROUND

The brain is part of the central nervous system (CNS) and is made up of two basic cell types: neurons and glia, wherein neurons are the main cell type in the brain responsible for message transmission. Neurons use electrical impulses and chemical signals to transmit information between different areas of the brain. They also transmit information between the brain and the rest of the CNS. The neurons that transmit messages from the CNS to other parts of the body are called ‘efferent’ neurons. The neurons that transmit messages from the other parts of the body to the CNS are called ‘afferent’ neurons. The neurons that relay messages between neurons in the CNS are called interneurons. When neurons carry signals from one place to another in the CNS they may also be referred to as ‘relay’ neurons. The neuron has a cell body, called a Soma, which contains the cell's nucleus and receives information. Thin filament dendrites extend outwardly from the Soma and act to carry information from other neurons to the Soma. The neuron also has a long tail called an Axon that extends from the Soma and terminates with a number of synapses. The Axon carries information from the Soma to the synapses. In turn the synapses are used for connecting to dendrites of other neurons.

Relay neurons in the brain, as well as other neurons, transmit messages by relaying a signal received from other neurons. If a neuron receives a large number of inputs from other neurons, Once the number of inputs exceeds a threshold, the neuron is triggered to send an impulse along its axon. This is called an ‘action potential’.

At the large scale e.g., cortical brain regions, brain states, and the transitions between them, are typically supported through a dynamic balance between excitation and inhibition. This balance is termed excitability. When the overall balance is disturbed, a network or region can become hyper-excitable, which can lead to pathological states of the brain, such as seizures. The efficacy of treatment depends on a complex interplay between structural and functional network structures, local neural dynamics, and endogenous rhythms (e.g., sleep or hormone dynamics).

Epilepsy is one of the most common neurological conditions affecting over 65 million people worldwide. Epilepsy is characterized by the tendency to have spontaneous seizures. A seizure may take many different forms including: ‘Absence seizures’ where a person becomes unconscious for a short time; ‘Focal aware seizures’ where the person is conscious and will usually know that something strange is happening; ‘Focal impaired awareness seizures’ where a person's consciousness is affected and they may be confused; ‘Myclonic seizures’ which are muscle jerks; ‘Tonic and atonic seizures’ where a person's muscles suddenly become stiff; ‘Tonic clonic seizures’ where a person becomes unconscious, their body goes stiff and they jerk and shake as their muscles relax and tighten rhythmically. In some cases, the cause of seizures is readily apparent (e.g., a brain tumour or cortical lesion); however, for the majority, the definitive cause is unknown.

From a clinical standpoint, doctors will typically evaluate people that have experienced a seizure-like event or symptoms often associated with seizures and epilepsy, and the EEG is often used to look for abnormalities that are strongly correlated with epilepsy and seizures. Such data are typically examined by a medical professional to detect the presence of epileptiform discharges (EDs). Epileptiform discharges are transient waveforms typically lasting for several tens to hundreds of milliseconds. They may be divided into a number of types.

Seizures are typically believed to be the result of disruptions in the level of neuronal excitability. In particular, mechanisms that govern the normal balance between excitation and inhibition can become compromised causing parts of the brain to become hyperexcitable. Hyperexcitability can be characterised at different scales. For example, at the cellular level it is strongly associated with the so-called paroxysmal depolarization shift (PDS) of cortical pyramidal cells. At the macroscale, it manifests in pathological electrical activity, captured using electroencephalography (EEG), called epileptiform discharges (EDs). EDs can be thought of as an umbrella term that encompasses both interictal (i.e., between seizures) epileptiform activity (e.g., spikes) as well as ictal activity (i.e., seizures).

Recent studies have presented evidence for underlying rhythmicity in EDs. Although such cycles have been shown to follow several temporal scales including ultradian, circadian, multidien and even circannual rhythms, relatively little is currently known about the mechanisms governing these rhythms and how intrinsic and extrinsic factors can modulate the likelihood of EDs. This limits the extent to which this knowledge of rhythmicity can be used for clinical benefit.

Over one third of people with epilepsy are considered refractory: they do not respond to drug treatments. For this significant cohort of people, surgery is a potentially transformative treatment. Brain surgery is a potentially life-changing treatment for people with epilepsy who do not respond to drug therapy. For those people with epilepsy for whom surgery is considered appropriate, long-term seizure freedom is achieved in around 50% of cases. However, success rates may be as high as 80% where an affected brain region is clearly identifiable but as low as 15% in cases where no such brain region is apparent. Many people with epilepsy display a reduction in seizure rates immediately after surgery; however, their seizures often return over time and may be different in nature to those with which they were initially diagnosed. With seizures occurring with or without surgery, there is therefore a desire to monitor a persons' brain to determine or predict upcoming seizures.

Identifying brain regions responsible for seizure generation and spread is complex and so the number of people considered suitable for surgery is relatively low and outcomes are non-optimal. Several computational methods that combine network analysis and mathematical modelling have been proposed to support surgical planning by evaluating virtually the potential impacts of the surgical resection. In such models, representations of brain networks are extracted from clinical data. However, these methods typically consider brain networks to be static after surgery.

Clinically, brain networks can be characterized through structural or functional relationships. Structural connections essentially represent the anatomical links between brain regions as typically measured using magnetic resonance imaging (MRI). These structural links are hypothesized to form the basis of functional connections between brain areas. Typically, functional connections are inferred statistically from timeseries data such as functional MRI, electroencephalography (EEG), or magnetoencephalography (MEG).

Leandro Junges et al, in “Epilepsy surgery: Evaluating robustness using dynamic network models” Chaos 30, 113106 (2020); describes a dynamic network model of seizure transition to systematically evaluate the influence of the network structure in seizure propensity before and after virtual resections.

WO2013182848 describes a system adapted to assist with assessing susceptibility to epilepsy and/or epileptic seizures in a patient, the system including: a device configured to receive patient brain data; a device configured to generate a network model from the received patient brain data, wherein nodes in the network model correspond to brain regions of the patient brain data and connections between the nodes of the network model correspond to measured connections between the brain regions; a device configured to generate synthetic brain activity data in at least some of the nodes of the network model; a device configured to compute seizure frequency from the synthetic brain activity data by monitoring transitions from non-seizure states to seizures states in at least some of the nodes over time; a device configured to use the seizure frequency to compute a likelihood of susceptibility to epilepsy and/or epileptic seizures in the patient, and a device configured to compare the computed likelihood with another likelihood of susceptibility to epilepsy and/or epileptic seizures in order to assess whether the likelihood has increased or decreased.

In general, an EEG is a test used to measure electrical activity of a brain, in particular a human brain. An EEG may be used to determine abnormalities in neural activity. Existing EEG testing uses electrodes located onto a patient's scalp. These electrodes may be referred to as scalp electrodes. The electrodes may comprise of small metal discs with thin wires that are pasted onto a person's scalp. The electrodes detect electrical charges that result from the activity of brain cells, in particular large groups of brain cells.

Different systems exist for electrode placement. One such system is the standard ‘10-10’ system, however a preferred system is the ‘10-20’ system. The relative positions of scalp electrodes for existing EEG testing conform to the 10-20 international system. The 10-20 system comprises different types of electrodes associated with different lobes, including: Fp (frontopolar), F (frontal), C (central), P (parietal), O (occipital) and T (temporal). A further electrode type is A (for the ears). Each lobe-electrode in the 1-20 system is labelled with one of the above types followed by one of: I) an even number representative of the electrode being placed on the right hemisphere of the brain II) an odd number representative of the electrode being placed on the right hemisphere of the brain; III) ‘z’ representing the electrode being placed on the midline.

This 10-20 system is shown in FIG. 1 wherein twenty-one electrodes labelled Fp1, Fp2, F7, F3, Fz, F4, F8, A1, T3, C3, Cz, C4, T4, A2, T5, P3, Pz, P4, T6, O1, O2 are applied to a user's scalp 2. The 10- 20 system nomenclature is applied when looking on top of the user's head wherein the nose (nasion) 4 is at the top of the picture near the electrodes Fp1 and Fp2 whilst the back of the head (Inion) 6 is at the bottom of the picture near electrodes O1 and O2. The midline 7 extends between the nasion 4 and inion 6, upon which is located electrodes Pz, Cz and Fz. The two ears 8a, 8b are linked by a further line 9 upon which is situated the electrodes T3, C3, Cz, C4, T4.

Diagnosing epilepsy typically combines EEG findings with the patient's case history and other neurological findings to come to a diagnosis. Existing EEG caps typically include all of these electrodes, and clinicians manually analyse the available readings from all electrodes to make a diagnosis. Making such a cap with every one of the listed 21 electrodes of the ‘10-20’ system may be time consuming and may be relatively expensive because of the need to manufacture a cap with all the 21 electrodes. Furthermore, a standard 10-20 system cap with all the electrodes may suffer from wastage due to post manufacture testing prior to sale wherein a defect on one of the 10-20 electrodes may result in the cap being disposed of or being fixed. Furthermore, if just one of the listed 10-20 electrodes is out of position beyond a particular tolerance, then the cap may also need to be adjusted prior to sale or use.

CN102499674 discusses that epilepsy diagnosis may be based on EEG and that existing ways of fixing electrodes makes electrode positions deviate greatly resulting in inaccurate multi-lead EEG acquisition. CN102499674 goes onto describe an electroencephalogram cap for “multi-ensuring contacts in close contact with the scalp”. The cap in CN102499674 uses a socket structure wherein the electrode holders are fixed on the main body according to electrode positions F3, F4, C3, C4, P3, P4, O1 and O2. CN102499674 describes that electrode positions F3, F4, C3, C4, P3, P4, O1 and O2 are commonly used potentials that include the main functional areas of the brain.

WO2013056099 describes apparatus for determining probabilistic measures of seizure activity (PMSA) values based on a plurality of seizure detection algorithms and/or body signals used as inputs for the seizure detection algorithms.

US20070149952 describes systems and methods for managing intake of a pharmaceutical agent. The systems and methods are for controlling intake of an antiepileptic drug wherein one or more signals from a patient are processed to predict an onset of a seizure.

WO2022104412 describes a system using a combination of EEG sensors, biochemical sensors, photoplethy smothgraphy (PPG) sensors, microphones and accelerometers to detect, predict and/or classify various physiological events and/or conditions related to epilepsy, sleep apnea and/or vestibular disorders. The events can include neuro-electrical events.

US20070250133 describes systems and methods for detecting and/or treating nervous system disorders, such as seizures. Certain embodiments relate generally to implantable medical devices (IMDs) adapted to detect and treat nervous system disorders in patients with an IMD. Certain embodiments include detection of seizures based upon comparisons of long-term and short-term representations of physiological signals.

US20160206236 describes systems and methods for managing epilepsy. US20160206236 describes to characterises a patient's propensity for a future epileptic seizure and communicates to the patient and/or a health care provider a therapy recommendation.

### SUMMARY

In a first aspect there is presented a system for estimating a likelihood of epileptiform activity, over time, of a patient; the system comprising a processor and a memory; the memory storing a model for estimating the likelihood of epileptiform activity of the patient; the model being configured to use: I) data associated with at least one physiological factor; II) data representing coupled brain activity for a plurality of different brain regions of the patient; the processor configured to: III) fit the model to at least a first value associated with likelihood of epileptiform activity derived from one or more measurements of the patient's brain activity; IV) estimate the likelihood of epileptiform activity, over time, from the fitted model.

The system of the first aspect may be modified in any suitable way described elsewhere herein including but not limited to any one of the following options.

The system may therefore provide an estimate of the likelihood of epileptiform activity by using a computer model associated with the brain wherein the model determines a plurality of values representing brain activity for a brain region. The likelihood may comprise a time dependent risk function. The model may estimate the future likelihood of epileptiform activity. The patient may be a human or another animal. The coupled brain activity data may be derived from a model of the patient's brain, which may be derived from EEG measurements. Measurements of epileptiform activity typically occur over a time period. Epileptiform activity may include seizures and interictal epileptiform discharges (that do not have a clinical manifestation but are markers of epilepsy). In some examples the activity measured is interictal activity. The system may be configured to output a control signal based on the estimation. The control signal for controlling the output of an alert. The alert may be an audible alarm, a haptic effect, a communication to a remote device or user. The system may be used for prognostic potential. The estimation of epileptiform activity may be compared to data associated with a treatment the patient has received; is receiving or is about to receive. The system may be used to determine an estimate of a seizure likelihood after a given treatment (e.g., AED). From the comparison, the system may further be configured to provide an objective indication as to whether the medication has the desired effect (e.g., lowering the seizure likelihood). The system may comprise a plurality of processors or devices for implementing the steps above. Each step may be implemented by a different device. Alternatively, a common device may implement a plurality of the steps. The system may comprise one or more interfaces for receiving sets of physiological data and/or any brain activity data.

The system may further comprise one or more wearables for measuring physiological data and outputting one or more signals to one of the interfaces. The system may further comprise one or more brain data measuring devices for measuring brain activity data and outputting one or more signals to one of the interfaces. The memory may further store any one or more of the; fitted versions of the model, brain activity data and physiological data. The memory may also be used for storing other patient data such as patient details (name, age, address, medical records, history of medicines and treatments for epilepsy, etc).

The system (and associated method/s) may be for estimating a likelihood of future epileptiform activity. Optionally, the data representing coupled brain activity comprises data from a brain network model. Optionally, the one or more measurements of the patient's brain activity are measurement from EEG scalp electrodes. Optionally the one or more measurements is single set of a readings of electrodes.

The system may be configured such that the one or more measurements of the patient's brain activity comprises data associated with interictal activity. The system may be configured such that the one or more measurements of the patient's brain activity are measurements of interictal activity. Optionally, the one or more measurements of the patient's brain activity comprises interictal related data (or otherwise known as non-ictal or non-seizure data). Optionally the model may be generated by the processor. Optionally the model may be generated using. Optionally the system may estimate the likelihood of future epileptiform activity without using ictal-based data (or ‘non-ictal’) based data.

Optionally, the system may update the estimation. Optionally, the system may be configured such that the processor updates the fitted model using at least any one or more of:
a) further one or more measurements of the patient's brain activity;
b) further data associated with at least one physiological factor.

Optionally, the further one or more measurements of the patient's brain activity comprise measurements taken after the fitting of the model. Optionally the further data associated with at least one physiological factor comprise measurements taken after the fitting of the model.

The system may be configured such that the processor generates, from the fitting of the model, a time dependent data profile, the said data in the profile associated with future epileptiform activity. The epileptiform activity may be associated with EEG data.

The system may be configured such that the data associated with at least one physiological factor comprises: a time-varying function associated with the physiological factor; and, a weighting; the processor configured to fit the model by varying the weighting and/or the time varying function.

The system may be configured such that the data associated with at least one physiological factor comprises data derived from measurements of at least that first physiological factor of the patient.

The system may be configured such that the data associated with at least one physiological factor comprises data derived from measurements of at least a first physiological factor of the patient. The system may be configured such that the data associated with at least one physiological factor further comprises data derived from measurements of a second physiological factor of the patient; the second physiological factor being different to the first physiological factor.

The system may be configured such that the data associated with one or more physiological factors comprises measurements of those physiological factors from the patient.

The model may use a plurality of different physiological data sets with the model, wherein each physiological data set comprises data derived from measurements, of a patient, of a different physiological factor. The model may be updated upon receiving a new set new physiological data. The updating of the model may include adapting, using the new data of the physiological factor, the time-varying function associated with the respective physiological parameter. Upon updating the said time varying function, the processor may create a further fitted version of the model using the said updated time varying function.

The system may receive patient physiological data from one or more devices. The said one or more devices may be external to the processor or form part of a system comprising the processor. The device may comprise a wearable configured to measure the physiological data and output one or more signals associated with the data, that are subsequently received and then used by the processor. The system may receive patient physiological data via an interface. The interface may form part of the aforesaid system. The system may receive brain activity data, from one or more brain activity monitoring devices, such as an EEG. The brain activity monitoring device may be external to the processor or form part of a system comprising the processor. The system may further comprise storing the fitted model in a memory.

The system may be configured such that the data associated with the physiological factor comprises any one or more of the following types: patient sleep data; patient stress data or data related to stress-related variables; patient cortisol data; patient blood glucose data.

Patient sleep data may include sleep stage data. Patient cortisol data may include cortisol concentration levels in interstitial fluid or other direct or indirect measures such as, but not limited to: indirect measure based upon correlations in heart rate variability, skin conductance and temperature. The physiological data may be any data that demonstrates a correlation to epileptic seizures.

The system may be configured such that the model describes: the dynamic activity of each of the said brain regions with respect to time; the excitability within each of the brain regions with respect to time.

The system may be configured such that the data representing coupled brain activity comprises data associated with the patient's brain derived from EEG measurements.

The system may be configured such that the data representing coupled brain activity comprises data from a brain network model describing functional connections between the different brain regions. The data representing coupled brain activity may comprise an adjacency matrix.

The system may be configured such that the first value associated with likelihood of epileptiform activity derived from one or more measurements of the patient's brain activity comprises data derived from EEG measurements of the patient's brain.

The system may be configured such that: the processor is configured to fit the model to at least: the first value associated with likelihood of epileptiform activity derived from one or more measurements of the patient's brain activity; a second value associated with likelihood of epileptiform activity derived from one or more further measurements of the patient's brain activity; wherein the measurements for the second value are taken after the measurements taken for the first value.

The system may be configured to: I) generate a first version of the model by fitting the output of the model to the first value, associated with likelihood of epileptiform activity derived from one or more measurements of the patient's brain activity; II) updating the model by generating a second version of the model by fitting the output of the model to the first and second values associated with likelihood of epileptiform activity derived from one or more measurements of the patient's brain activity.

The system may be configured such that the model comprises a set of two coupled stochastic differential equations for each considered brain region.

The system may be configured such that the model is based off a bifurcation structure describing the transition between background brain states and seizure-like brain states.

The bifurcation may be, for example, a Hopf bifurcation. The differential equations may be derivatives with respect to time. The equations may describe the time-evolution of one or more complex variables. The complex variable may be denoted as ‘zi’, where ‘i=1, . . . ,N’, that represents the N network nodes. The coupling between the differential equations may be linear and proportional to the difference in the node states. The bifurcation model may be based on a Hopf bifurcation exemplified by equations 2 and 3 listed elsewhere herein. A Hopf bifurcation may be suitable for modelling epileptic seizures because it because it allows for a transition between two different dynamical regimes (e.g. a healthy background state and a seizure-like state). The bifurcation model may further include one or more variables associated with excitability of a node. The time variation, preferably slow time variation, of the excitability variable may be represented by one of the differential equations.

Further associated with the first aspect there is presented a method for estimating a likelihood of epileptiform activity, over time, of a patient; the method using a memory storing a model for estimating the likelihood of epileptiform activity of the patient; the model being configured to use: I) data associated with at least one physiological factor; II) data representing coupled brain activity for a plurality of different brain regions of the patient; the method using a processor for: III) fitting the model to at least a first value associated with likelihood of epileptiform activity derived from one or more measurements of the patient's brain activity; IV) estimating the likelihood of epileptiform activity, over time, from the fitted model.

The method may be configured to utilise similar features and implement similar steps as described for the system in the first aspect. There is also presented a non-transitory computer readable medium comprising processor-executable instructions that when executed by said processor give effect to the method as described above.

There is further presented, in a second aspect, an apparatus for use in determining epileptiform activity of a patient; the apparatus comprising a plurality of electrodes for contacting the patient's head; the plurality of electrodes comprising between four and upto, and including, 18 electrodes.

The apparatus may be modified according to any suitable way described elsewhere herein, including but not limited to any one or more of the following options.

The apparatus may be configured such that each of the electrodes corresponds to an electrode placement position of the 10-20 system.

The apparatus may be configured such that: at least one electrode whose location covers the frontal left hemisphere, at least one electrode whose location covers the frontal right hemisphere, at least one electrode whose location covers the occipital left hemisphere, at least one electrode whose location covers the occipital right hemisphere.

The apparatus may be configured such that the plurality of electrodes comprises a minimum of six electrodes wherein: at least one electrode is an FP1 or FP2 electrode; at least one electrode is an T electrode; at least one electrode is an C electrode; at least one electrode is an O electrode; at least one electrode is the Fz electrode; at least one electrode is the Cz electrode.

The apparatus may be configured such that the plurality of electrodes comprises a minimum of eight electrodes wherein: at least one electrode is the FP1 electrode; at least one electrode is the FP2 electrode; at least one electrode is the T3 electrode; at least one electrode is the C3 electrode; at least one electrode is the T4 electrode; at least one electrode is the C4 electrode; at least one electrode is the O1 electrode; at least one electrode is the O2 electrode.

The apparatus may be configured such that: at least one electrode is the Fz electrode, which, in use, acts as a ground electrode; at least one electrode is the Cz electrode, which, in use, acts as a reference electrode.

The apparatus may be configured such that the electrodes are dry electrodes.

There is also presented a kit of parts comprising a system as described in the first aspect and an apparatus as described in the second aspect.

In a further aspect there is presented a system for estimating the likelihood of epileptiform activity, over time, of a patient; the system comprising at least one processor and configured to:
A) receive patient physiological data; and,
B) using the processor, use the said received patient physiological data with a model, to output one or more values of brain activity for at least one brain region; the model relating physiological data to brain activity;
C) forecast epileptiform activity, over time, of the patient based on the output one or more values of brain activity.

The system may be modified according to any teaching herein, including but not limited to any of the optional features listed above for the first aspect and/or any one or more of the following options.

The system may be configured such that the model comprises a set of two coupled stochastic differential equations for each considered brain region.

The system may be configured such that the model is based off a bifurcation structure describing the transition between background brain states and seizure-like brain states.

The system may be configured such that the patient physiological data comprises any of:
I) patient sleep data;
II) patient cortisol data;
III) patient blood glucose data.

The system may further comprise a device for generating the patient physiological data. The device may be a wearable.

In a third aspect there is presented a system for estimating the change over time of a likelihood of future epileptiform activity of a patient; the system comprising a processor and a memory; the memory storing a model for estimating the likelihood of epileptiform activity of the patient; the model being configured to use data representing coupled brain activity for a plurality of different brain regions of the patient; the coupled brain activity associated with a brain network; the processor configured to: I) determine one or more parameters for the model using one or more measurements of at least one physiological factor of the patient; II) fit the model, using the one or more parameters, to at least a first value associated with likelihood of epileptiform activity derived from one or more measurements of the patient's brain activity; the one or more measurements of the patient's brain activity for determining the data representing coupled brain activity in the model; III) estimate the change over time of the likelihood of future epileptiform activity from the fitted model.

Associated with the third aspect, there is presented a method for estimating the change over time of a likelihood of epileptiform activity of a patient; the method using a memory storing a model for estimating the likelihood of epileptiform activity of the patient; the model being configured to use data representing coupled brain activity for a plurality of different brain regions of the patient; the coupled brain activity associated with a brain network; the method using a processor for: I) determining one or more parameters for the model using one or more measurements of at least one physiological factor of the patient; II) fitting the model, using the one or more parameters, to at least a first value associated with likelihood of epileptiform activity derived from one or more measurements of the patient's brain activity; the one or more measurements of the patient's brain activity for determining the data representing coupled brain activity in the model; III) estimating the change over time of the likelihood of future epileptiform activity from the fitted model.

The method and/or system of the third aspect may be adapted according to any teaching herein, including but not limited to any of the optional features described for the first aspect.

### BRIEF DESCRIPTION OF DRAWINGS

Embodiments of the present invention will now be described in detail with reference to the accompanying drawings, in which:

FIG. 1 shows the 10-20 system of the prior art;

FIG. 2 shows a schematic example of a computer device for implementing the method described herein;

FIG. 3a shows the number of EDs from the 107 subjects with epilepsy;

FIG. 3b shows a boxplot showing basic sample statistics of the number of EDs (showing minimum, lower quartile, median, upper quartile and maximum);

FIG. 3c shows the normalised rate of EDs per hour;

FIG. 4 shows normalised ED rate for two groups (1 and 2) of people when investigating circadian distribution of EDs;

FIG. 5 shows different distributions of sleep onset and offset;

FIG. 6 shows models vs measured ED activity for two different groups;

FIG. 7 shows models vs measured ED activity for group 1;

FIG. 8 shows models vs measured ED activity for group 2 taking into account blood plasma glucose;

FIG. 9 shows an example arrangement of electrodes;

FIG. 10 shows an example method for estimating a likelihood of epileptiform activity of a patient.

### DETAILED DESCRIPTION

There now follows a non-limiting example of a method for determining propensity to epilepsy. This example may be computer implemented. The method may be implemented by a system comprising one or a plurality of modules or devices to give effect to the method. In the following discussions of different examples reference is made to the ‘patient’, i.e., the person using the system to monitor their own seizure likelihood, however this patient may also be referred to as the ‘person’ or ‘user’. Collectively the monitored factors inform the generation of computer estimates of future seizure likelihood over different timescales. One potential application of the system is in prognosis, where the system is used to estimate seizure likelihood over long timescales of e.g., months or years. Another application of the system is in forecasting, where the system is used to estimate seizure likelihood over short timescales of, e.g., hours or days. A patient is typically a human but may be another animal such as a dog or a cat. Furthermore, a further person such as a medical professional or a vet, may use the system with the patient. In the examples underneath it is understood that the method and system are applied for the purposes of estimating seizure likelihood. Such estimating may include, but is not limited to: diagnosis, forecasting and/or prognosis, for example, assessing the effect of a treatment on the seizure likelihood.

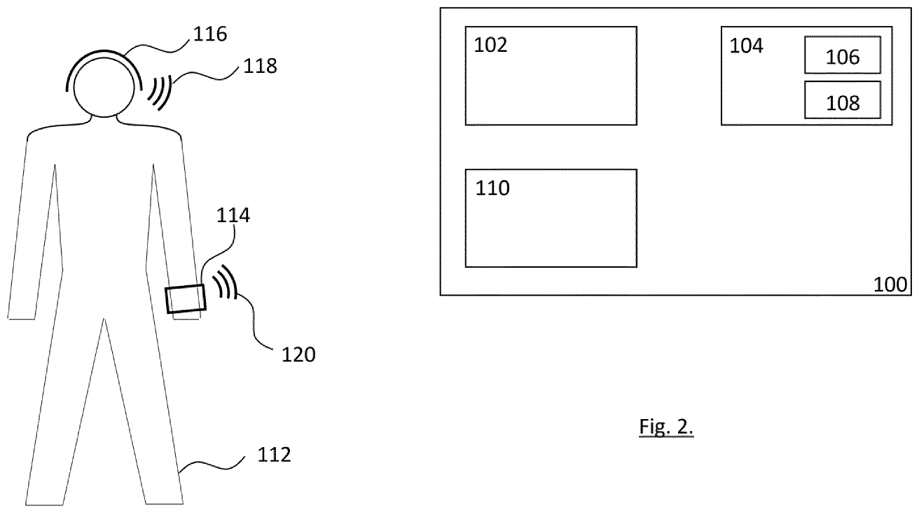

An example of the system and its relationship to the user is shown in FIG. 2. A computer device 100 comprising a processor 102 and a memory 104 is used to implement the method. The memory 104 may have one or more separate modules 106 or memory allocations for storing instructions to perform the method. The memory 104 may have one or more separate modules 108 or memory allocations for storing data according to the method. This data may be any of, but not limited to: patient brain data, such as from an EEG; a brain network model as calculated by the method or pre-established and stored in the memory module 108; output data from the method. The computer device 100 may also comprise one or more interfaces for receiving one or more external devices, such as but not limited to any of: an EEG headset 116 placeable on a user 112; a wearable 114, such as a watch, for measuring a physiological state or otherwise output physiological information associated with the patient 112. The interface may also receive data from other devices and may communicate with external devices by being able to transmit data to external devices and receive data from external devices. FIG. 2 shows that the headset 116 and the wearable 114 transmit data to the interface 110 via wireless electromagnetic communications, however other forms of data transfer may be used include wired electrical signals or optical signals transmitted in free-space or via a waveguide such as an optical fibre. The computer device 100 may be adapted using other examples of computer apparatus herein. The computer device may be, or form part of, for example, a smart phone, tablet computer, smart watch, desktop computer. In an alternative example the computer device may comprise a device similar in function to the wearable for outputting physiological data associated with the patient 112. In another example the computer device may form part of the headset 116. The computer device or another external device may have any of: a haptic actuator; a visual alarm system; an audible alarm system that may be triggered to activate with one or more control signals output by the computer device 100 in response to the output of the method. For example, the computer device 100 may determine that the patient may be due to have a seizure and may send an electronic signal to the interface 100 to output a control signal to a remote device, such as the wearable, to vibrate on the patient's wrist to alert the patient accordingly. The example of FIG. 2 may be applied to any other examples described herein.

A person uses an EEG headset with a plurality of electrodes to establish a set of electrical readings from a plurality of the electrodes. This is typically done over a time period of 5 minutes to 1 hour, however any time period can be used. During this time period the person is not having an epileptic seizure. One or more such sets of readings may be taken and from those sets, one or more are selected based upon whether or not the person has or does not have a seizure. In other words, only sets of data are used where the person did not experience a seizure during the measurement duration. The resultant electrical readings from the electrodes are used to generate a representation of the persons functional brain network structure. The term ‘functional’ here meaning the activity of one region of the brain monitored by one electrode in relation to another region of the brain monitored by another electrode. The network structure is represented using an adjacency matrix ‘A’, also any other mathematical representation other than an adjacency matrix may be used. The adjacency matrix is formed by comparing the electrical signals and determining any relationships in brain activity The comparison may be done using a correlation. The determination may involve quantifying relationships in brain activity.

In an example, if four EEG electrodes are used then four regions of the brain are being monitored wherein an interictal ‘spike’ is an increase in potential, typically followed by a corresponding decrease beyond a threshold potential value. A ‘base level’ is the nominal potential reading having one or more values existing underneath the aforesaid ‘threshold’. The threshold defining an interictal ‘spike’ may be a single threshold value, in some examples there may be multiple threshold values denoting potential levels for different interictal spike strengths. The readings are taken over twenty minutes and:
electrode 1 only outputs a base level of signal throughout all the twenty minutes
electrodes 2 and 3 both output three raised potential spikes at: a) ten minutes forty seconds; b) fifteen minutes and two seconds; and, c) nineteen minutes and fifty-one seconds;
electrode 4 output one raised potential spike at two minutes three second.

The comparison determines that electrodes two and three are coupled or ‘connected’. This comparison may be done by correlation, such as correlating the time-series of the different electrodes to reveal that there is a connection between electrodes 2 and 3 since they display similar types of activity around similar times. When linking the brain areas and activity to a graph theory perspective, electrodes one, two, three and four are nodes of the brain network and the connection between nodes two and three is a single graph edge.

This adjacency matrix may otherwise be formed using other techniques including using fMRI data and MEG data.

In principle, at its simplest level, the adjacency matrix may be determined from one or more readings from each of two or more EEG electrodes. Thus, the adjacency matrix may be formed from a single electrical signal from each of multiple electrodes such that, that single time snapshot is used to see what brain regions display similar types of activity, for example by simultaneously increasing. In principle the adjacency matrix may be formed from two or more electrodes. Preferably the EEG signal include many readings from many electrodes, for example 100 or more readings. The number of electrodes used may be four or more, preferable between 4-20, optionally between 4-10. The EEG electrodes may use a standard electrode-placement system such as the 10-20 system.

Once an adjacency matrix has been derived from the EEG recording, the method then utilises this within a dynamic model in order to estimate the propensity to seizures and epilepsy. This may be a propensity to epilepsy perse or propensity to one or more seizures.

The specific model used may vary, however in this example the method uses a modified form of the normal form of the subcritical Hopf bifurcation. Furthermore, this model has a separate equation describing the time evolution of one of the primary variables that collectively describe excitability within the node. The model therefore provides a set of two coupled stochastic differential equations for each brain region (node).

The inputs to this model include the adjacency matrix described above as well as data corresponding to at least one, preferably both of: a) the person's cortisol level (‘CORT’); and b) the person's sleep stages during relevant periods. Other physiological data may be used as well including blood glucose data. The data for the persons cortisol level is related to stress and may be monitored in a number of ways including wearables, blood sample test results. Sleep data may be monitored in various ways including using a set of EEG electrodes or another sleep monitor such as, but not limited to a wearable. The sleep and cortisol data are described further underneath. Initially, the excitability component of the model may start with physiological data derived from the patient, or, alternatively, defaults from other sources such as physiological data from one or more other people, for example a plurality of other people. Using other people's physiological data may be used when the patient's physiological data is not yet available to input into the model. The model may be updated, over time, to include further physiological data, for example by replacing other people's physiological data or updating the patient's own physiological data.

A person's sleep behaviours and/or sleep quality and/or sleep patterns and a person's stress levels (which may be represented by cortisol level), as well as other physiological factors discussed elsewhere herein, are influential factors in timing and occurrence of seizures and epileptiform activity.

Equations 1 and 2 shows an example of the coupled equations used wherein equation 1 describes zⱼ (t), the dynamics of the j^{th} node over time, whilst λⱼ(t) represents the excitability of node j over time. The second portion of equation 1, having the term starting with “β/N”, factors in the coupling of different regions of the brain.

$$\frac{{dz}_{j}}{dt} = {z}_{j} ( {λ}_{j} - 1 + i ω + 2 {\vert {z}_{j} \vert}^{2} - {\vert {z}_{j} \vert}^{4} ) + \frac{β}{N} {\sum }_{k = 1}^{N} {A}_{kj} ( {z}_{k} - {z}_{j} ) + α {dW}_{j} \qquad [ Equ . 1 ]$$

$$τ \frac{d {λ}_{j}}{dt} = {λ}_{j 0} + {( {λ}_{EXT} )}_{j} - {λ}_{j} - {\vert {z}_{j} \vert}^{2} \qquad [ Equ . 2 ]$$

In the above equations:
Aₖⱼ is the adjacency matrix. It is a parameter input to the model and is typically kept constant throughout an instance of use of the model to calculate a seizure propensity. This may be a weighted matrix or binary (unweighted). In this example it is binary and takes the value of 0 or 1 wherein 1 is a connection between two nodes k and j and 0 is no connection between the two nodes k and j.
N is the number of nodes (brain regions) in the network. This is typically determined by the number of electrodes used in the EEG recording. It is a parameter input to the model and is typical kept constant throughout an instance of use of the model to calculate a seizure propensity. An example range is 3-100 nodes, although other ranges and node numbers may be used.
λⱼ₀ is the baseline level of excitability also known as λ_{BASE}. It is a parameter input to the model and is typical kept constant throughout an instance of use of the model to calculate a seizure propensity. This data may be provided from clinical data or based off an estimation.
λ_{TEXT} is the external perturbation to the excitability and is associated with the sleep and/or cortisol readings (hence may be a function of those readings and may vary in time). It is a parameter input to the model and may be kept constant throughout an instance of use of the model to calculate a seizure propensity. This data may be provided from clinical data, based off an estimation or from readings from physiological parameter monitors (such as wearables) as discussed elsewhere herein.
β is the global coupling strength between nodes. It may have a typical range between 0.06 and 6, although other ranges and values may be used. It is a parameter input to the model and is typical kept constant throughout an instance of use of the model to calculate a seizure propensity. This data may be provided from clinical data or based off an estimation.
ω is the frequency of the stable limit cycle, and corresponds to the dominant frequency observed during seizure-like activity. It may have a typical range between 3 and 50 Hz, although other frequency ranges and frequency values may be used. It is a parameter input to the model and is typically kept constant throughout an instance of use of the model to calculate a seizure propensity. This data may be provided from clinical data or based off an estimation.
α is noise strength. It may have a typical range between 0.005 and 0.10 although other ranges and values may be used. It is a parameter input to the model and is typically kept constant throughout an instance of use of the model to calculate a seizure propensity. This data may be provided from clinical data or based off an estimation.
τ is the timescale of the variable λ It may have a typical range between 5 and 50 seconds, although other ranges and values may be used. This parameter is typically constant throughout an instance of use of the model to calculate a seizure propensity. The data of this may be provided from clinical data or based off an estimation.
Wⱼ is a Complex Weiner process, typically of dimension 2.

In one non-limiting example the parameters for the models are given below in Table 1.

TABLE 1

| Parameter | Value |
|---|---|
| ω | 20 rad/s |
| β | 0.35 |
| α | 0.055 |
| τ | 3 s |
| λ_{BASE} | 0.65 |

There may be a number of ways to use the above model including, but not limited to, the type of data used; the methods used to collect the data; how the data is used in the model.

In this example, different data is used for λ_{EXT}. This λ_{EXT} variable may be a function of one or more physiological data sets, such as but not limited to, sleep data and cortisol data. Other data sets may be, for example, glucose, alcohol levels, anxiety, hours of sleep one or two nights prior to seizure, menses, medication regimens or missed medications, other methods of measuring anxiety, or other measurable trigger factors for seizures such as but not limited to those discussed in Haut, S. R., Hall, C. B., Masur, J. and Lipton, R. B., 2007; Seizure occurrence: precipitants and prediction; Neurology, 69 (20), pp.1905-1910, which is incorporated herein by reference. Each physiological data set forms a component of the overall λ_{EXT} variable, wherein each component may comprise a time dependent profile and a weighting coefficient. The time dependent profile, such as λ_{EXT, CORT}, may be represented by a mathematical function mapping the physiological value to changes in excitability over time. The function may extend over any time period, for example 12 hours, 24 hours or greater than 24 hours. Patient physiological data may be used to generate this function. The weighting may be a coefficient to the original profile. For example, when the method uses two physiological factors of sleep and cortisolλ_{EXT} may be represented by the following equation:

$${λ}_{EXT} = {p}_{s} {λ}_{EXT , SLEEP} + {p}_{c} {λ}_{EXT , CORT} \qquad [ Equ . 3 ]$$

Wherein λ_{EXT,SLEEP} is a time-dependent data profile for sleep stage, λ_{EXT, CORT} is a time-dependent data profile for cortisol level, p_{S} is the coefficient for the sleep stage, p_{C} is the coefficient for the cortisol level. Other physiological factors may be represented in a similar manner with a time-dependent data profile and associated coefficient. Each such factor may be incorporated into the λ_{EXT} variable, for example by summing them as shown in equation 3, however other forms of representing λ_{EXT} may be used. The λ_{EXT} variable may incorporate one or a plurality of such physiological factor wherein each physiological factor may be represented by one or more variables and optionally one or more respective variable coefficients. In one example λ_{EXT} in equation 3 may be changed to further add a glucose factor p_{G}×λ_{EXT, GLUC}; wherein p_{G} is the glucose-related coefficient and λ_{EXT, GLUC} is a time-dependent data profile associated with blood glucose. Each of the respective terms such as ‘pₛλ_{EXT, SLEEP}’ may be referred to elsewhere as a ‘model variable’ and different versions may correspond to different values of the coefficient and/or different values of the profile.

As described above, the functions such as λ_{EXT, CORT}, may start as default functions established from general population data for the physiological parameter. For example, the average person's Cortisol levels over a 24-hour period, in a sample of 50 people. The model may use these initial default functions to make a computed estimate of future seizure likelihood. Once measurements are made of the patient's actual physiological data, these can then be used to update the profile. For example, the patient physiological data may be used to update the entire profile or a part of it.

In this example sleep data may be monitored by the patient wearing a set of EEG electrodes for 24 hours, or wearing a smart-watch. Other time periods can also be used. The terms ‘sleep cycle’ and ‘sleep stage’ may be used interchangeably in the following discussion. The electrode signals in EEG, or actigraphy from the smart-watch, are used to determine the stages of the patients sleep cycle throughout the time of wearing the electrodes. In other examples the stages of sleep cycle are derived from other input data such as a device that uses a microphone to monitor the sounds a person makes whilst they are sleeping and processing the data to determine the sleep cycle. This processing can be by comparing sounds to stored sound data and/or using machine learning. The EEG may record the sleep stages and ED for the patient during the same period of use.

The data is used to provide sleep stage times, in other words the relative length of different sleep stages in a typical overnight sleeping period. There may be at least two different sleep stages. Preferably three or more sleep stages are used, preferably four or five sleep stages are used. The sleep stages may be any one or more of, but not limited to: wake; N1 (stage 1); N2 (stage 2); N3 (stage 3); REM sleep (Rapid Eye Movement). The data are further used to provide details of sleep onset and offset, as well as quality of sleep as measured by the amount of time spent in each sleep stage.

The sleep stage data comprises a time varying profile of patient sleep stage. This data is represented by the λ_{EXT, SLEEP} component of the λ_{EXT} variable. The time varying profile may comprise a time duration and/or time value for each stage of sleep. These stages are provided in more detail below wherein features, such as, but not limited to, proportion of wave types, associated with any of the stages may be used as data in the said model. The first stage may be the wake stage or stage W, which further depends on whether the eyes are open or closed. There may then follow an N1 (Stage 1) is the lightest stage of sleep and starts when more than 50% of the alpha waves are replaced with low-amplitude mixed-frequency (LAMF) activity. There may then follow an N2 (Stage 2) representing deeper sleep as your heart rate and body temperate drop. There may then follow an N3 (Stage 3), considered the deepest stage of sleep and is characterized by a much slower frequency with high amplitude signals known as delta waves. There may further be a REM Sleep stage associated with dreaming.

In this example cortisol data is also monitored by the patient using an electrochemical cortisol sensor. This sensor may determine a cortisol level from body fluids in vitro and/or from wearable sensors. Fluid enabling cortisol determination may be, but not limited to: saliva, blood, sweat, interstitial fluids, urine, tears. Hair may also be used to determine cortisol. The sensor outputs data associated with cortisol levels over time which may be used to form a time dependent cortisol level profile of the patient that forms the data set for, λ_{EXT, CORT}.

The model may be used initially to calculate and output a time varying function of seizure likelihood. The extent of time that the function may extend over may be any length of time, for example over a 12-hour period, a 24-hour period or longer. This model output may then be used to compare against one or more values of epileptiform data derived from measured brain activity or epileptiform data, such as one or more susceptibilities to future epileptiform activity as determined using the system described in WO2013/182848, which is incorporated herein by reference. The measured data may take other forms such as data from a seizure diary. The data from the EEG headset may be used to calculate the one or more susceptibilities to future epileptiform activity. The EEG headset may be a standard 10-20 headset but is preferably a headset with fewer electrodes than a standard 10-20 EEG headset, but still having the electrodes placed in accordance with the 10-20 positioning system. Using an EEG with fewer electrodes entails that the headset may be simpler and easy to use, hence usable by a patient without the need for a medical practitioner. For example, the headset may be used by the patient at home. Examples of such a headset are described elsewhere herein. For purposes of further discussion, the measured data values of susceptibilities to epileptiform activity are simply referred to as “EEG-based risk readings”, although as discussed above, other measurement data may be used to determine the risk readings. The time of day and/or the date of the reading are typically recorded and used so that a computer system 100 such as that shown in FIG. 2, may compare the ‘EEG-based risk readings’ to the values of seizure risk output by the model given its current model parameter values.

If the comparison determines that models output risk estimation is different to one or more of the EEG-based risk readings then computer system 100 updates the model by updating one or more of the parameters, such as the ‘p’ coefficients in equation 3. This process is now described below.

The model in Equations 1 and 2, and utilising equation 3 are then used to determine the best fit of the model to the ED data. To determine a best fit, each different time-dependent value of ‘z’ over the period is converted to an ED likelihood, for example by comparing to a threshold value. The model is run a plurality of times wherein each time, at least one of the coefficients p_{S}, p_{C} is changed. The new values of p_{S}, p_{C} (or other coefficients such as p_{G} (if glucose is used as a factor)) that produce an estimated (modelled) risk-time-profile that best match the EEG-based risk readings are determined to be the most appropriate coefficients for that person. The coefficients represent how much each physiological variable are correlated to a person's EEG-based risk reading. For example, for some people sleep may not be determining factor, but cortisol is, in which case the p_{S} coefficient will be low or zero whilst the p_{C} coefficient will be relatively high. The coefficients may take any range including but not limited to the range 0-1.

The output from running the above model is a determination of ‘z’ for each node. In the discussion above, values of ‘z’ for each time segment may be converted to an estimated ED by calculating an average value of ‘z’ from all the nodes ‘j’. For example, the mean of the ‘z’ values may be taken and compared to a threshold value for ‘z’. Other analyses are also possible.

Once the optimal values of coefficients p_{S}, p_{C} are established, the person may then monitor further cortisol and sleep data and use it with the stored model to update the respective function such as λ_{EXT, CORT}, etc. Once this new physiological data is used to update the λ-function, the model may be re-run to establish a better fit to the EEG-based risk readings. The model may also be re-run to fit the EEG based risk readings each time a new EEG-based risk reading is taken. For example, the model may initially adapt the ‘p’ coefficients in equation 3 once the patient inputs a first EEG-based risk reading a time t=1. The model determines, for the best fit, that p_{C} is 0.25 whilst p_{S} is 0.75. The patient then inputs a second EEG-based risk reading a time t=2. The model determines, from fitting to both the first and second EEG-based risk readings that p_{C} is 0.35 whilst p_{S} is 0.65. Thus, the model fits a function estimating seizure likelihood to one or more EEG-based risk readings that are typically separated in time. The EEG-based risk readings that are used to fit the model are typically single values taken at different times of the day but may in principle be one or more sets of continues risk readings (for example if the patient uses EEG apparatus for an extensive period of time).

The model's output may be used to estimate the likelihood of an epileptic seizure and/or identify periods during which epileptiform activity is more likely and may get progressively more accurate:
A) the more EEG-based measurement readings are taken (and fitted to);
B) the more updated the patient's physiological data is, hence how accurate the λ-function is for each particular physiological parameter.

Each time one of the above new readings A)/B) are taken, the model may be re-run to establish a better fit.

The EEG measurements used to generate EEG-based risk readings may be for any duration of time, for example, of up to any of: 5 minutes, 10 minutes, 20 minutes. Such a short time duration to make a reading, that is used to improves the models' risk estimation profile, is advantageous because a patient may make such an EEG reading quickly.

The system and method may therefore present a better forecaster or identifier of future epilepsy or epileptic seizure risk. This may be from being more accurate in determining how seizure likelihood varies over time. When compared to traditional ways of determining likelihood wherein a clinician looks at multiple EEG readings, the system and method may require less time. Furthermore, the system and method may at least partially automate the determination of a seizure compared to traditional ways of seizure prediction. In some examples, readings from a reduced number of EEG electrodes (compared to using the full complement of electrodes of the 10-20 system) may be used to determine the brain network. Using a reduced number of electrodes, hence readings, may give rise to a more efficient system in that less calculations may be need, thus making the detection system faster.

The output may be used to create an output signal such as an electrical signal. This signal may be used to control a device or component either within the system used to enact the above method or an external device. For example, the signal may be a control signal send to an alarm or to a haptic actuator to signal to the person or a medical professional that the person is likely going to have a seizure.

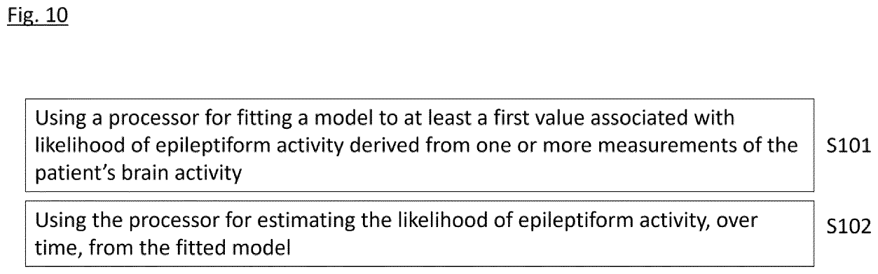

The model may be developed using a method as shown in FIG. 10 wherein: step S101 uses a processor for fitting the model to at least a first value associated with likelihood of epileptiform activity derived from one or more measurements of the patient's brain activity; and step S102 uses the processor for estimating the likelihood of epileptiform activity, over time, from the fitted model.

This above example may be adapted in any way including, but not limited to:
The method of obtaining the brain network including the data used.
The duration of time taken to record EEG readings.
The need to generate the brain network. In other examples the brain network may already be provided.
The dynamic model used.
The model parameters used. For example, some of the parameters in the equation may be removed such as, but not limited to the base level of excitability, the value B/N.
Any of the parameter values.

Any feature of the above method and of the ways it can be varied and adapted may be used with other examples herein.

### FURTHER EXAMPLES

There now follows an example of the system and method. This example may be adapted with features of other examples disclosed herein including but not limited to any of: type of data collected to develop the mathematical brain network; the device or devices used to collect brain data; the devices used to collect other data such as but not limited to CORT data and/or sleep data; the number and frequency of collection of any of the aforesaid data; the techniques used to develop the brain network; the techniques used to model the dynamic behaviour; the device or system type that the method is implemented on and the device or system components; the outputs of the system and method. Furthermore, any of the above features used in this example may be used in other examples.

In this example a computer system is used to implement the features or steps. This computer system may be a portable computer system such as a smart phone but may be other types of computer system such as a desktop computer or even a cloud computer system or any other type of computer system described herein. The computer system has one or more memory components upon which is stored computer readable instructions for implementing the features or steps presented herein. The different steps may be modules such as software modules, additionally or alternatively features may be implemented as dedicated hardware modules.

In the following example, reference is made to what the ‘method’ or the ‘system’ does, however it should be understood that a step made in reference to the method may equally be implemented be a feature of the equivalent system and vice versa.

A non-limiting overview to guide the reader to the method described underneath is as follows:
A) Firstly, a patient is furnished with a device for measuring brain data (such as an EEG) as well as devices for measuring other physiological data (such as sleep, stress or glucose)
B) Secondly, parameters of a dynamic computational model are determined using inputs from the available devices. For example, a brain network structure is formed using the signals from the device measuring brain data (such as an EEG). Parameters corresponding to perturbing factors, such as sleep and CORT are formed using the signals from the appropriate device.
C) Thirdly, the method applies the dynamic computational model to create synthetic brain activity data that describes transitions from interictal to ictal states over time.
D) Fourthly, a dynamic measure of seizure likelihood is determined from the generated synthetic data. This dynamic measure may involve, for example, computing the regularity of synthetic EDs over a specified time window.
E) Finally, the dynamic computational model and subsequently the measure of seizure likelihood may be updated over time by recalculating the relevant parameters from additional data collected at future instances of time and following the process described in C and D.

In the following discussion reference is made to ‘brain network structure’ however the term ‘brain network’ may equally be used to mean the same thing. To mathematically form the brain network, EEG data of a person's brain is used when the person is not having a seizure. Other brain data that may be used in the alternative or in addition to the EEG data may be MRI data from an MRI scanner or MEG data. In general, any brain data recording device may be used.

A patient (also referred to herein as a user) puts on an EEG headset and (if required) activates the headset to monitor brain activity and output electrical signals representing that said activity. The patient would normally do this in periods where no seizures are occurring, i.e., an interictal state. An EEG headset outputs a plurality of electrical signals that are received by the computer system. These signals represent measurement of brain activity localised to brain regions underlying the location of each electrode. Data may be collected over varying periods of time. The EEG headset may, in some examples, form part of the system.

The network structure may be determined in different ways. The readings are used to estimate the functional connectivity. The output of the steps forming the brain network structure may be an adjacency matrix. This adjacency matrix may be a topological adjacency matrix. The adjacency matrix may be used in the dynamical computational model.

A brain network may be represented by a graph having a plurality of nodes and edges connecting the nodes. The nodes topologically represent different areas of the brain, such as those proximal to where an EEG electrode is positioned. A graph may be represented by an adjacency matrix wherein a connection between two nodes I, j is represented by the matrix element (i,j). If the network is a binary network, then those matrix elements are usually either a value of ‘1’ for a connection existing or ‘0’ for no connect existing. In weighted networks, a connection between two nodes is a value typically in the range [0,1], for example, 0.65.

In general, the computer 100 may generate a network model based on patient brain data. In some non-limiting examples, the structure of this model is inferred from the patient data using the method of beta weights described in ‘Benjamin O, Fitzgerald T H B, Ashwin P et al, A phenomenological model of seizure initiation suggests network structure may explain seizure frequency in epilepsy. J Math Neuroscience 2012, Vol. 2, No. 1, pp. 1-41’ (the contents of which are hereby incorporated by reference). In other examples, the computer may additionally, or alternatively, use another measure of nonlinear correlation. In general, the model comprises a set of nodes that correspond to brain regions of the patient brain data and connections between the nodes of the network data structure correspond to connections between the brain regions, as recorded in the source brain data. In the case of EEG data, for instance, the brain regions will correspond to regions of the brain close to the electrodes of the EEG headset.

The brain network may be a set of nodes that correspond to brain regions of the patient brain data. Connections between the nodes of the network data structure correspond to connections between the brain regions, as recorded in the source brain data. In the case of EEG data, for instance, the brain regions will correspond to regions of the brain close to the electrodes of the EEG machine. The connections in the brain network in the examples herein are functional network connections rather than structural network connections.

A patient's physiological state, including factors such as, but not limited to: sleep and stress may further be used with a brain network to determine a dynamic measure of future seizure likelihood over different periods of time. Sleep quality and duration may be measured using a watch or other wearable device. Cortisol, a hormone which drives the human body's physiological response to stress, may be measured directly from interstitial fluid collect from a device worn on the waist, or indirectly through variability in heart rate, temperature and skin conductance as measured from a watch (or other device). Other factors may include glucose, alcohol etc as previously described. These data may be of differing durations and collected at different instances of time. For example, EEG data, using a headset such as one described herein which has less electrodes than a standard 10-20 system, may be collected at 8 am for 10 minutes, sleep data may be taken from the previous night over 8 hours, cortisol data for 20 minutes and blood glucose data at a single time point. These examples should not be considered limiting and other durations may be used. Ideally, they would be collected within close proximity to each other.

The data from the EEG and the physiological data, is uploaded to a computer device such as the device 100 in FIG. 2. This computing device then develops the dynamic model as described above with respect to equations 1, 2, 3. These inputs are used to personalise model parameters, relative to their original functional form. The original functional form may be determined from historic data from other patients, or through knowledge of the impact of the given physiological factor on seizure likelihood.'

The dynamic model is then used to generate synthetic data from which the dynamic measure of seizure likelihood is determined. For example, if the person's susceptibility to EDs with respect to cortisol is high, then the model may output a high seizure likelihood at times when cortisol levels are high. In another example, if sleep quality or duration were poor, then the seizure likelihood may be elevated until the next period of sleep.

At any future time afterwards, for example later in the same day, or the next day or the next week; the user may collect additional data as described above. For example, the user may choose to collect EEG data twice a day, or to record blood glucose immediately prior to and following a meal or intense exercise. These further recordings are then used to re-factor the generated dynamic model so update the dynamic measure of seizure likelihood.

Depending on the purpose, the output may be a display of information or an alert such as a haptic vibration or an audible alarm. The user may then take any appropriate response to this forecast including taking medicine or seeking other medical help. By getting new updated estimates of the seizure-likelihood, the patient and caregiver may get an idea of the overall effect of a medication or treatment on the seizure-likelihood (i.e. whether it is having the desired effect of lowering the seizure-likelihood), as well as an understanding of which factors (e.g. alcohol, sleep, stress) have the greatest impact on their personal seizure likelihood.

There now follows further optional details concerning the dynamic computation models usable in methods and systems described herein.

In general, the computer may generate synthetic brain activity data based on at least some of the nodes of a network model. The dynamic computational model may be based on features of human seizure data. A model may be used that phenomenologically and/or physiologically models transitions from interictal to ictal states in brain regions over time. Additionally, or alternatively the synthetic brain activity data may be generated using a probabilistic model, for example, representation by a Markov process.

The computer may optionally determine one or more seizure likelihoods from the synthetic brain activity data generation. This may be done by monitoring transitions from non-seizure states to seizures states in at least some of the nodes over time.

Additionally, or alternatively, an analytic technique based upon the escape time of the chosen network model may be used.

The computer may use different definitions of seizure rate. This may be any of: the rate of transition of a specific node within the network model; an average of transitions across all nodes of the network model; an average across all nodes of the time spent in the dynamic region corresponding to the seizure state of the model per unit of simulation time. For the latter option, an example may be summing the transitions observed in a time-series and dividing this by the duration of the brain activity simulation.

The computer 100 may use the seizure frequency computed to provide an estimate of the seizure likelihood, which can be relevant in terms of assessing overall susceptibility to epilepsy/future epileptiform activity in the patient. It is to be understood that one or more standard statistical techniques may be used to do this.

The computer may compare the computed likelihood with another value. When computing a ‘likelihood’, the other value may comprise a likelihood of susceptibility to epilepsy or epileptic seizures. For example, a likelihood value computed using the above process performed on previous data obtained from the same patient. This comparison may then performed/repeated at a later point in time. This later point in time may be after a period of taking a new anti-seizure medication (ASM) or anti-epileptic drug (AED), in order to assess the efficacy of the medication for example, giving an indication of whether the susceptibility to epilepsy/seizures of the patient (or group of patients) has increased or decreased compared to the original baseline measurement.

Example evidence is now provided underneath demonstrating the applicability of physiological data such as stress, blood glucose and sleep in helping estimate propensity for epilepsy.

### Example Study of Two Physiological Factors

The example study is firstly summarised and then described in more detail underneath the summary.

EDs from two groups of patients (Group 1 and Group 2) were determined from 107 patients over a 24-hour period. Patients that showed above average ED activity during the sleep hours were grouped into Group 1 whilst the remaining were grouped into Group 2. For Group 1, sleep-stage data was used as the λ_{EXT} perturbation profile in equation 2, also referred to as “λ_{EXT, SLEEP}”. Equations 1 and 2 were used to determine a time-profile of ‘z’ values over the same 24-hour period, which in turn were used to estimate EDs. To estimate EDs the output number ‘z’ (per time segment) was compared to a threshold value of 0.5 wherein if z>0.5 then this corresponded to an ED and z<0.5 corresponded to no ED. The model was run (instantiated) a plurality of times, wherein each instance the impact of the “λ_{EXT, SLEEP}” on the ‘z’ profile was varied by weighting the profile using the coefficient p_{S}. The perturbation time-profile “λ_{EXT, SLEEP}” was a time varying profile of sleep stage. In this study the sleep stage data was a representation of a person's average sleep stage time-profile from a different group of 77 healthy participants. In this analysis it was assumed that the average sleep stage time-profile was reasonably representative of the 107 patients. Each run of the model to fit the data used a different coefficient p_{S} for “λ_{EXT, SLEEP}” that varied from 0-1. In other words: a coefficient of 0 meant that the profile “λ_{EXT, SLEEP}” had no effect but a coefficient of 1 meant that the “λ_{EXT, SLEEP}” profile had maximum effect. The model instance with the best fit to the ED data corresponded to a particular coefficient p_{S} of “λ_{EXT, SLEEP}”.

A similar process of determining λ_{EXT, CORT} was used wherein an average person's daily CORT profile data was obtained from a different six healthy adults. This profile was used as the λ_{EXT} variable in equations 1 and 2 such that λ_{EXT}=λ_{EXT, CORT}. The CORT levels of average people rise and fall throughout the day and the established profile was used to investigate the relationship between CORT and actual EDs assuming the 107 people that monitored their EDs would have a similar distribution of CORT levels to the measured profile, hence a similar CORT time-profile. In a similar way to establishing the impact of sleep stages, the dynamic model was run through a plurality of instances wherein each instance varied the λ_{EXT, CORT} coefficient between 0 and 1.

Thus, in one set of analyses, the λ_{EXT} parameter factored in sleep but not CORT for Group 1; and in another set of analyses the λ_{EXT} parameter factored in CORT but not sleep for Group 2.

In a further set of analyses λ_{EXT} factored in CORT and sleep to model the EDs of group 1 and Group 2, In these analyses λ_{EXT} was the summation of two λ_{EXT} time-profile variables, one for sleep, one for CORT, with each variable having their own coefficient, as represented the form shown in equation 3. The model was run varying both the coefficients p_{S} and p_{C} wherein the best fit curve, determined by comparing the curve to the actual ED data represented a particular value of both coefficients.

The output of this study showed that both sleep and CORT may factor into a dynamic model to successfully map onto real measured ED data.

Thus, a similar process may be used to model a single patients ED profile if patient ED data is used. The inventors also realised that models other than that given by equations 1-3 may be used to determine the same relationships. A model therefore can be developed using one or more sets of physiological data about the patient, for example one set of data may be patients CORT, another may be sleep. One or more instances of the model may be run to establish the respective coefficients (such as p_{S} and p_{C}) of λ_{EXT} that fit the persons ED profile. After doing this, the model is then at a state of development such that it may be further used to forecast epileptic activity (although the model may be further developed by running further instances with new data such as new patient data for the same physiological factors and/or new data for different physiological factors). After model development, the patient may then monitor their sleep or CORT at a future time period. The future measured levels may be used to determine a likelihood of a seizure or forecast other epileptic activity. This may be done in different ways including any of (for example): comparing the CORT level with a corresponding CORT level of the previously derived data and identifying whether that level gave rise to an ED.

This study is now discussed in more detail underneath.

In one example study, the potential contribution of physiological factors was assessed by considering 24-hour profiles of epileptiform discharges from a cohort of 107 people with epilepsy. Two dominant subgroups with common distributions of epileptiform discharges (EDs) were found. This data was further interrogated using a mathematical model that describes the transitions between background and epileptiform activity in large-scale brain networks. This model was extended to include a time-dependent forcing term, where the excitability of nodes within the network can be modulated by other factors. This forcing term was calibrated using independently collected human cortisol recordings and sleep-staged EEG from healthy human participants. What was found in this example study, was that that either the dynamics of cortisol alone, or a combination of sleep stage transition and cortisol, could explain most of the observed distributions of epileptiform discharges (EDs).

The study confirmed that a dynamic computational model can accurately forecast periods of enhanced seizure likelihood relating to common triggers such as stress and stressful situations as well as sleep, sleep deprivation and fatigue.

The mammalian stress-response is driven by circulating glucocorticoid hormones: predominantly cortisol in humans and corticosterone in rodents, herein ‘CORT’. Ultradian and circadian rhythms of CORT are controlled by the hypothalamic-pituitary-adrenal (HPA) axis, a neuroendocrine axis, wherein a delayed negative-feedback loop mediates hormones secreted from the pituitary and adrenal glands.

To explore possible contributing factors underpinning these different mechanisms, the inventors developed a mathematical modelling framework that: a) describes transitions between background states and EDs; b) relates excitability to the likelihood of these transitions; c) considers the impact of intrinsic and extrinsic factors on excitability. Model parameters were calibrated using independently collected 24-hour hormone profiles from six healthy participants and sleep staged polysomnography data from forty-two healthy participants. Synthetic minority oversampling was used to account for discrepancies in group size and generated synthetic distributions of EDs. The goodness of fit between these model derived distributions and those observed in the cohort of people with IGE, was explored. The mathematical analysis revealed evidence to support the view that the likelihood of EDs is modulated by both transitions in sleep stages, as well as by ultradian fluctuations in cycling CORT levels.

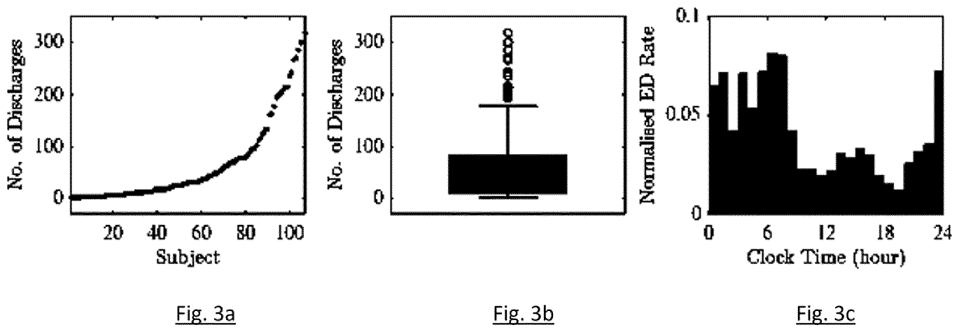

FIG. 3a shows the number of EDs from the 107 subjects with epilepsy. FIG. 3b shows a boxplot showing basic sample statistics of the number of EDs (showing minimum, lower quartile, median, upper quartile and maximum). FIG. 3c shows the normalised rate of EDs per hour. This data has been previously presented by Seneviratne, U., Boston, R. C., Cook, M. an'D'Souza, W., 2016. Temporal patterns of epileptiform discharges in genetic generalized epilepsies. Epilepsy & Behavior, 64, pp.18-25. Descriptions of the data set for FIGS. 3a-3c can be found in: Seneviratne, U., Hepworth, G., Cook, M. and D′Souza, W., 2016. Atypical EEG abnormalities in genetic generalized epilepsies. Clinical Neurophysiology, 127 (1), pp.214-220.

In the study, the median number of EDs over 24 hours was approximately 29, although several individuals had more than 200 events as shown in FIG. 3b. Examination of normalised ED patterns on an hourly basis suggested that the likelihood of EDs varied across the day as shown in FIG. 3c. In other words, for each individual, the number of EDs at each hour was divided by their total number of EDs and then normalised over the cohort.

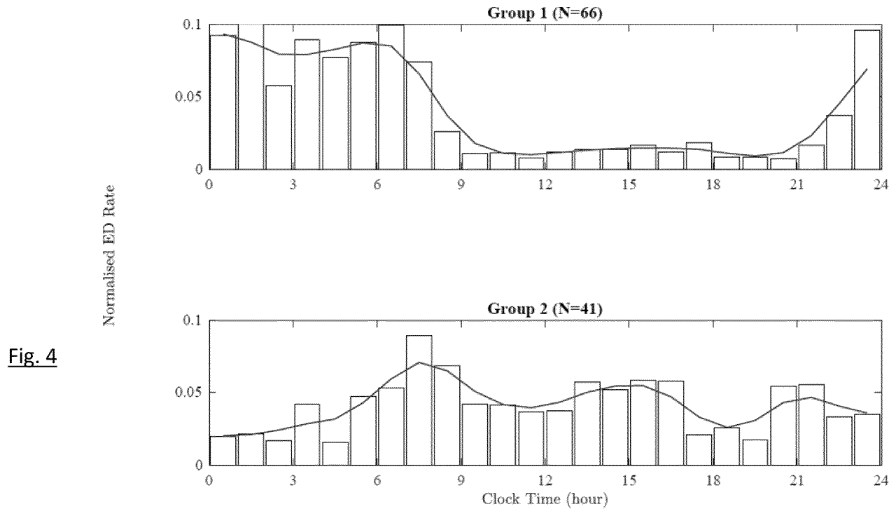

FIG. 4 shows normalised ED rate for two groups (1 and 2) of people when investigating circadian distribution of EDs. Group 1 had 66 individuals and Group 2 had 41 individuals. To investigate the possible circadian distribution of EDs, similarities were considered between subjects. The cross-correlation coefficients of time series representing the individual hourly ED rate were computed using MATLAB®. This led to a correlation matrix C, with entries Cij corresponding to the similarity between the pattern of EDs in subject I and in subject j. The closer the value of Cij to 1, the more similar the distribution of EDs of subject i and subject j. Subsequently, subjects were clustered according to their correlation coefficients using k-means clustering and the Calinski-Harabasz criterion to optimise the number of clusters. This analysis revealed two primary subgroups within the overall cohort of people with IGE that displayed different circadian ED distribution patterns.

Candidate mechanisms were explored that could explain differences in ED distributions between the two sub-groups identified by the cluster analysis. The (empirical) likelihood of EDs in Group 1 (FIG. 4, top) displayed a significant increase in the propensity for EDs during the night and lower levels during daytime. In contrast, the likelihood of EDs in Group 2 (FIG. 4, bottom) displayed greater variation during waking hours.

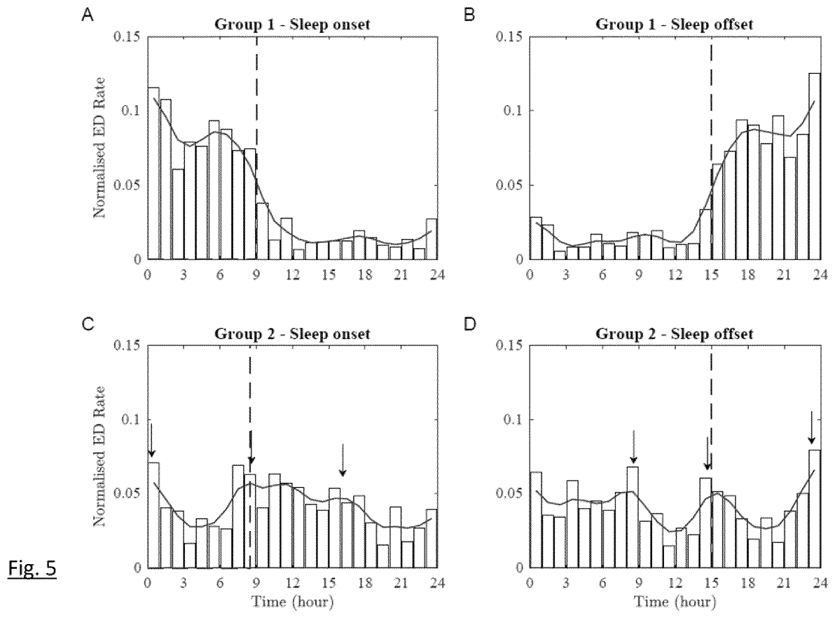

To assess the impact of timing of sleep and its duration on ED distributions, time was adjusted within each subject such that t=0 corresponded to either their sleep onset or sleep offset. The resulting distributions are presented in FIG. 5 Panels A-D. For Group 1, the ED rate was higher for approximately 9 hours starting at habitual sleep onset (Panel A), while it was relatively low during the rest of the day. In Panel B, the same trend was observed but shifted to the 9 hours before waking. For Group 2 (FIG. 5, panels C, D) no increased levels of EDs were found during sleep, instead the distribution suggests a potential daytime ultradian rhythm.

To quantify this more explicitly, the parameter Fi in equation 4, (i=1, 2) was introduced to measure the fraction of EDs occurring during sleep for each group:

$${F}_{i} = \frac{1}{{N}_{i}} {\sum }_{j = 1}^{{N}_{i}} \frac{{ED}_{S , j}}{{ED}_{tot , j}} \qquad [ Equ . 4 ]$$

Here, Nᵢ was the number of subjects in Group I; ED_{S,j} and ED_{tot,j} are the numbers of EDs for the j^{th} subject in the group occurring during sleep time and across the full 24-hour period, respectively. It was found that F1=0:8, suggesting that 80% of EDs in Group 1 were clustered during the sleep period. In contrast F2 was 0.37, suggesting that in Group 2 just over a third of discharges occur during the sleep period, consistently with the 8-9 hour sleep time (i.e., a third of 24-hour). Interestingly, for Group 2 three peaks of similar height were found around 8 hours prior to sleep, sleep onset, and sleep offset (Panel C). The equivalent pattern was found when aligning by sleep offset (Panel D).

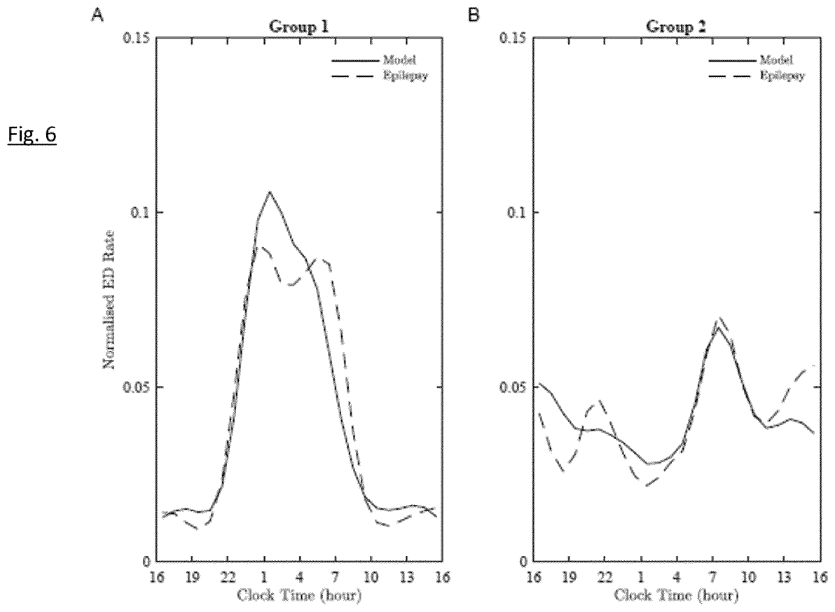

The model used in Equations 1 and 2 was then used to simulate the ED rate and compared against the data from FIG. 5. The comparison is shown in FIG. 6 wherein FIG. 6 panel ‘A’ shows a good fit, in Group 1 (only taking into account sleep with λ_{EXT}), between the model (solid line) and the measured ED data (dashed line). FIG. 6 panel B shows similar comparison results for group 2 wherein only CORT is taken into account with λ_{EXT}.

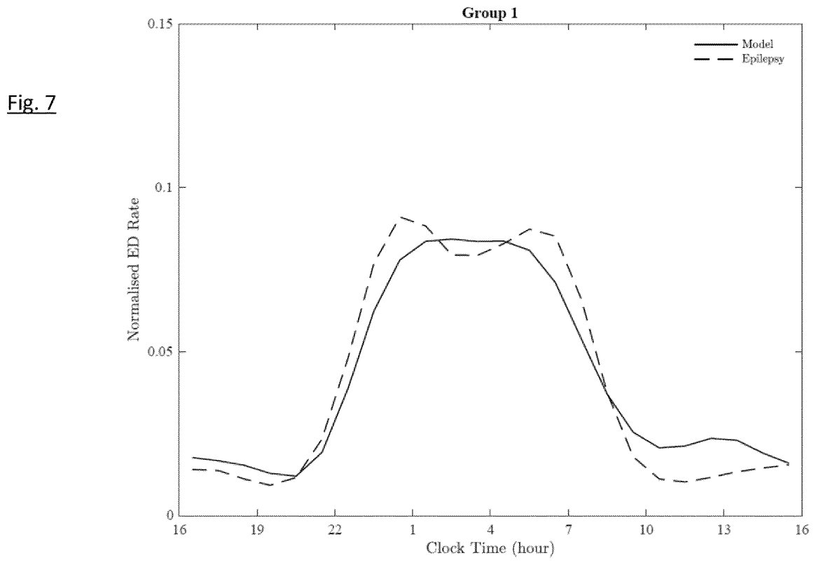

In the figure for Group 1, sleep staged polysomnography data was collected from healthy controls as an external input to the excitability of the model λₑₓₜ. FIG. 7 shows, for Group 1, the predictions of the model for a virtual cohort of 66 individuals (solid line) compared with the observed ED distributions for that group (dashed line).

The model predicted a sharp increase in ED occurring during the first part of the sleep period, followed by a sharp decrease in the morning. The slow reduction in the number of EDs during the night is consistent with the findings that the NREM is predominant during the first part of the sleep, while REM is predominant during the second half. Although the model captured most of the Group 1 ED variability (R=0.81), it failed to capture the bimodal distribution in ED rate shown in the overnight data. It further failed to capture daytime variability in the ED rate, suggesting the presence of at least a second mechanism governing the ED propensity.

In FIG. 6 for Group 2, levels of CORT were measured from healthy controls over the course of 24 hours as an external input to the excitability of the model Next. The model prediction for a virtual cohort of 41 individuals is shown in FIG. 6 (solid line). Comparing the model result with the ED data (dashed line), the inventors found that the model captures the morning and afternoon peaks displayed by Group 2, although the latter occurs about an hour earlier in the model. The simulation, here, does not account for the evening peak around 21 hours. The overall variability explained by CORT in Group 2 is 11% (R²=0.11).

Combined mechanism: sleep and CORT.

For each group, candidate mechanisms were identified that could explain the majority of the observed distribution of EDs. However, they found that the model failed to capture some variability. For example, in Group 1 the bimodal distribution during sleep, as well as some variability during the day, was not fully explained by the model. They therefore explored how combining the mechanisms of sleep and CORT impacted the ED distribution. The strength of the influence of sleep and CORT is given by the parameters p_{S} and p_{C}, respectively. Each parameter can vary from 0 (no impact on ED occurrence) to 1.2 (strong impact on ED occurrence).

A residual sum of squares (RSS) was used to identify the best fit. In Group 1, the inventors found the best fit (lowest RSS values) is obtained when p_{S}, p_{C}>0. This result is consistent with the above previous observation that sleep can explain the overnight peaks in EDs, with the contribution of CORT explaining variability during the day. This result suggests the coexistence of the two mechanisms (sleep and CORT) in Group 1. FIG. 7 shows the ED data (dashed line) and model output (solid line) corresponding to the lowest RSS for group 1, wherein the model uses both sleep and CORT with p_{S}=0:3 and p_{C}=0:4. Combining these mechanisms increases the explained variability from 81% (only sleep) to 95% (R²=0:95).

Conversely, the lowest values of RSS in Group 2 are obtained when p_{S}=0 and p_{C}=0:4, suggesting that the best fit for group 2 is obtained when CORT is the sole mechanism considered in the model. The explained variability increases from 11% (p_{C}=1) to 66% (R₂=0.66).

Blood glucose.

As described above, the model did not improve when including sleep with group 2. The inventors therefore considered that other physiological factors may be used to factor into modelling ED activity. One of these factors was blood plasma glucose levels. To assess the impact of glucose in the ED occurrence, the inventors used measurements of plasma glucose levels from eleven healthy subjects to define the external input to the excitability of the model λ_{Gluc}.

The fraction of explained variability in Group 2 improved when the inventors considered the combined effect of glucose and CORT as per equation 5 below:

$${λ}_{EXT} = {p}_{G} {λ}_{EXT , GLUC} + {p}_{c} {λ}_{EXT , CORT} \qquad [ Equ . 5 ]$$

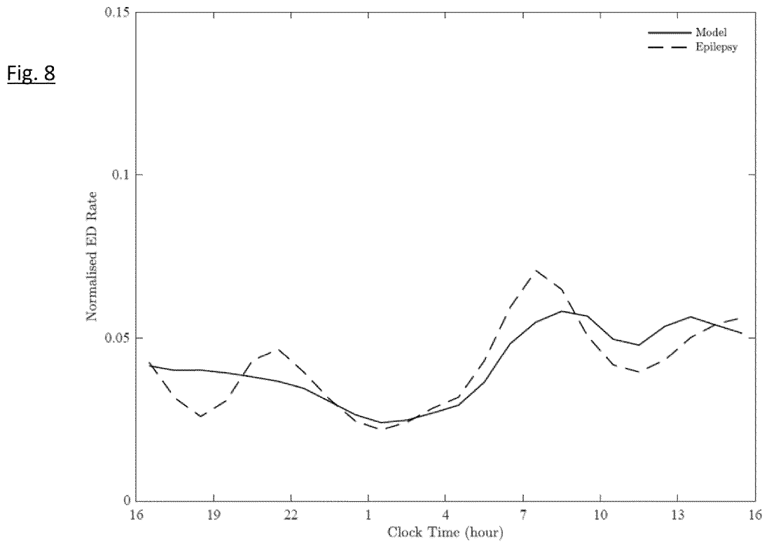

The minimum RSS over a grid of values of p_{G} and p_{C} was obtained when p_{G}=0:4 and p_{C}=1:8 (see FIG. 8 where the dashed line indicates ED measurements, and the solid line indicates the model). In this scenario, the percentage of explained variability is 68% (R2=0:68).

It is therefore shown that one or a combination of two or more physiological factors, such as but not limited to sleep, cortisol and blood glucose, may be used to model epileptiform activity and therefore may in turn be used to forecast such epileptiform activity. Other physiological factors may also be used with the model but are not included in the above example study.

### Example Apparatus with Electrodes for Monitoring Brain Activity

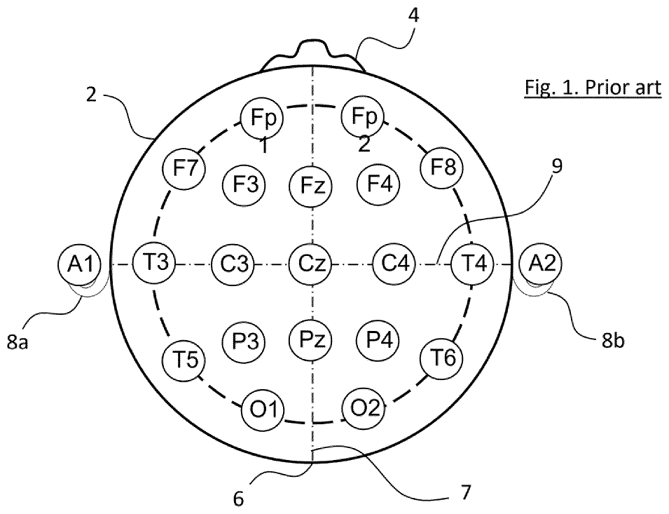

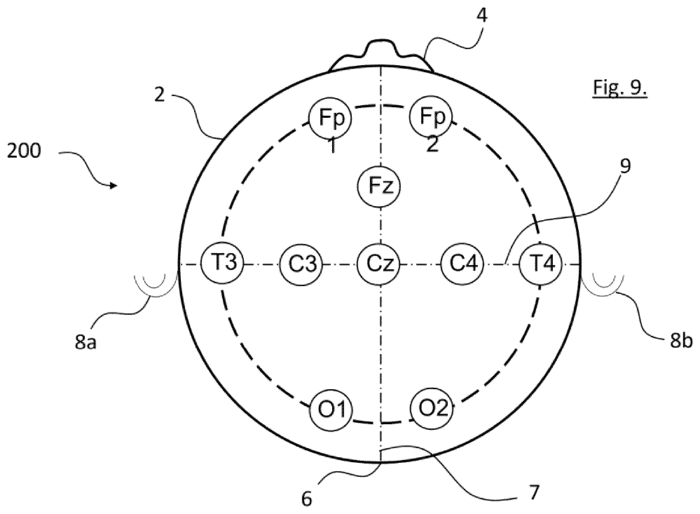

FIG. 9 shows an example of a set of electrodes 200 for use with an apparatus for measuring brain activity. Like references between FIG. 9 and FIG. 1 represent like features. The electrodes used are Fp1, Fp2, Fz, T3, C3, Cz, C4, T4, O1, O2, with respect to the 10-20 system of EEG electrode placement. Fz is used as a ground electrode whilst Cz is used as a reference electrode.

The apparatus may in principle have the plurality of electrodes comprising between four and A) upto, and including, 18 electrodes, or B) upto, and including, 18 electrodes.

The inventors have discovered that these electrodes, in particular when: at least one electrode is an FP electrode; at least one electrode is an T electrode; at least one electrode is an C electrode; at least one electrode is an O electrode; at least one electrode is the Fz electrode; at least one electrode is the Cz electrode; constitute a set of electrodes that may sufficiently map electrical activity of brain regions to an extent that may be used with the model described elsewhere herein. Additionally, or alternatively, the apparatus may be configured to have: at least one electrode whose location covers the frontal right hemisphere, at least one electrode whose location covers the occipital left hemisphere, at least one electrode whose location covers the occipital right hemisphere. Such a simple apparatus may be used to provide brain activity data for use with the model described herein.

The use with the model may be for developing the model and/or when estimating seizure likelihood using the developed model.

The apparatus may be an EEG apparatus. The electrodes used may be fewer or more electrodes, but less than the full complement of electrodes in the standard 10-20 system. Having fewer electrodes entails greater manufacturing simplicity and speed and ease of use when attaching electrodes to a person's head. Such a simple apparatus may allow a person to use it themselves without needing a clinician. The number of the 10-20 electrodes used may be in a range. The lower limit of any range may be any of: 4, 5, 6, 7, 8, 9, 10 electrodes wherein the upper limit of any range may be any of 10, 11, 12, 13, 14, 15, 16, 17, 18, 19, 20 electrodes, assuming the upper limit is always larger than the lower limit.

The electrodes are connected to wires that deliver electrical signals from the electrodes to further electrical apparatus for evaluating the electrical signals. Additionally, or alternatively the electrical signals output by the electrodes may be coupled to a wireless communications system that wirelessly transmit the electrical signals to the further electrical apparatus.

The apparatus may include one or more elements to attach the electrodes to, for example the apparatus may comprise a frame that is flexible or substantially rigid wherein the frame accommodates or otherwise holds the electrodes. The electrodes may be held by the apparatus using any connection mechanism such as but not limited to an adhesive. The apparatus may have a flexible web-like structure for laying over a person's head instead of a frame. The apparatus may have the electrodes either permanently attached or have the electrode be removable. The electrodes in some examples may be position-adjusted about a person's head to get the electrodes in the correct position. The apparatus may take the form of a headset or helmet and may be designed for a human or an animal, such as a pet (e.g., dog, cat).

Preferably the electrodes are dry electrodes so that they can be used by a patient. The dry electrodes may be formed from any suitable material including, but not limited to any of: tin, silver, sintered Ag/AgCl, disposable Ag/AgCl, gold, platinum and stainless steel. Dry electrodes also enable a quick use of the apparatus. A quick use of the apparatus may be desirable for a reference level measurement made by a user when using the developed model described above

### Examples of Computer Apparatus for Use with the System and Methods

The following describes optional examples of how the system and method may be implemented using computer hardware and/or software. Other examples of computer components described herein may equally be used with such examples described underneath.

Some portions of the above description present the features of the invention in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, the reference to these arrangements of operations in terms of modules should not be considered to imply a structural limitation and references to functional names is by way of illustration and does not infer a loss of generality.

Unless specifically stated otherwise as apparent from the description above, it is appreciated that throughout the description, discussions utilising terms such as “processing” or “identifying” or “determining” or “displaying “displayin”” or the like, refer to the action and processes of a computer system, or similar electronic computing device, that manipulates and transforms data represented as physical (electronic) quantities within the computer system memories or registers or other such information storage, transmission or display devices.

Certain aspects of the method or system include process steps and instructions described herein that may be in the form of an algorithm. It should be understood that the process steps, instructions, of the said method/system as described and claimed, may be executed by computer hardware operating under program control, and not mental steps performed by a human. Similarly, all of the types of data described and claimed may be stored in a computer readable storage medium operated by a computer system and are not simply disembodied abstract ideas.

The method/system may also relate to an apparatus for performing the operations herein. This apparatus may be specially constructed for the required purposes, or it may comprise a general-purpose computer selectively activated or reconfigured by a computer program stored on a computer readable medium that can be executed by the computer. Such a computer program is stored in a computer readable storage medium, such as, but not limited to, any type of disk including floppy disks, optical disks, CD-ROMs, magnetic-optical disks, read-only memories (ROMs), random access memories (RAMs), EPROMS, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

Any controller(s) referred to above may take any suitable form. For instance, the controller(s) may comprise processing circuitry, including the one or more processors, and the memory devices comprising a single memory unit or a plurality of memory units. The memory devices may store computer program instructions that, when loaded into processing circuitry, control the operation of the route provider and/or route requester. The computer program instructions may provide the logic and routines that enable the apparatus to perform the functionality described above. The computer program instructions may arrive at the apparatus via an electromagnetic carrier signal or be copied from a physical entity such as a computer program product, a non-volatile electronic memory device (e.g., flash memory) or a record medium such as a CD-ROM or DVD. Typically, the processor(s) of the controller(s) may be coupled to both volatile memory and non-volatile memory. The computer program may be stored in the non-volatile memory and may be executed by the processor(s) using the volatile memory for temporary storage of data or data and instructions. Examples of volatile memory include RAM, DRAM, SDRAM etc. Examples of non-volatile memory include ROM, PROM, EEPROM, flash memory, optical storage, magnetic storage, etc. The terms ‘memory’, ‘memory medium’ and ‘storage medium’ when used in this specification are intended to relate primarily to memory comprising both non-volatile memory and volatile memory unless the context implies otherwise, although the terms may also cover one or more volatile memories only, one or more non-volatile memories only, or one or more volatile memories and one or more non-volatile memories.

The algorithms and operations presented herein can be executed by any type or brand of computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. It is appreciated that a variety of programming languages may be used to implement the teachings of the invention as described herein.

In examples presented above, the model uses seizure risk data to compare against the output of the model (such as the model provided in equations 1-3). There now follows an example of a system for assessing susceptibility to epileptiform activity such as epilepsy or epileptic seizures, wherein the output data may be used to compare with the model's output. This system may have a plurality of devices for accomplishing different tasks. Each device may, in some circumstances perform a number of the tasks listed below. In some circumstances a single common device may accomplish all the tasks listed underneath and associated with different devices. The further optional details concerning the dynamic computation models, described above may be used for this system for assessing susceptibility to epileptiform activity.

The system for assessing susceptibility to epileptiform activity such as epilepsy or epileptic seizures may include a device configured to receive patient brain data. The system may include a device configured to generate a network model from the receive patient brain data. The nodes of the network model may correspond to brain regions of the patient brain data. Connections between the nodes of the network model may correspond to measured connections between the brain regions. The system may comprise a device configured to generate synthetic brain activity data in at least some of the nodes of the network model. The system may comprise a device configured to compute seizure frequency from the synthetic brain activity data by monitoring transitions from non-seizure states to seizure states in at least some of the nodes over time. This may be done using EEG data or other brain data. The system may further comprise a device configured to use the seizure frequency to compute a likelihood of susceptibility to epileptiform activity (such as epileptic seizures) in order to assess whether the likelihood has increased or decreased.

This above system may be used with any of the examples described herein. The above system may be part of the same system that generates and updates the model as presented in equations 1-3. As such, the overall system including the system described in the first aspect, may comprise devices to provide the functionality listed in the first aspect, such as, but not limited to any one or more of: one or more devices to receive physiological data, such as blood glucose data, etc; one or more devices to receive data related to patient brain activity; one or more devices to computationally generate the dynamic model described with respect to equations 1, 2, 3, and other similar models described herein; one or more devices to fit the model and estimate the likelihood of epileptiform activity, over time, from the fitted model.

1. A system for estimating the change over time of a likelihood of future epileptiform activity of a patient; the system comprising a processor and a memory;
the memory storing a model for estimating the likelihood of epileptiform activity of the patient; the model being configured to use data representing coupled brain activity for a plurality of different brain regions of the patient; the coupled brain activity associated with a brain network;
the processor configured to:
I) determine one or more parameters for the model using one or more measurements of at least one physiological factor of the patient;
II) fit the model, using the one or more parameters, to at least a first value associated with likelihood of epileptiform activity derived from one or more measurements of the patient's brain activity; the one or more measurements of the patient's brain activity for determining the data representing coupled brain activity in the model;
III) estimate the change over time of the likelihood of future epileptiform activity from the fitted model.

2. A system as claimed in claim 1 wherein the measurements comprise data associated with at least one physiological factor comprising:
a time-varying function associated with the physiological factor; and,
a weighting;
the processor configured to fit the model by varying the weighting and/or the time varying function.

3. A system as claimed in claim 1 wherein the measurements comprise data associated with one or more physiological factors comprising measurements of those physiological factors from the patient.

4. A system as claimed in claim 3 wherein the data associated with the physiological factor comprises any one of the following types:
I) patient sleep data;
II) patient stress data;
III) patient blood glucose data.

5. The system as claimed in claim 1 wherein the model describes:
I) the dynamic activity of each of the said brain regions with respect to time;
II) the excitability within each of the brain regions with respect to time.

6. The system as claimed in claim 1 wherein the data representing coupled brain activity comprises data associated with the patient's brain derived from EEG measurements.

7. The system as claimed in claim 6 wherein the data representing coupled brain activity comprises data from a brain network model describing functional connections between the different brain regions.

8. The system as claimed in claim 1 wherein the first value associated with likelihood of epileptiform activity derived from one or more measurements of the patient's brain activity comprises data derived from EEG measurements of the patient's brain.

9. The system as claimed in claim 1 wherein:
the processor is configured to fit the model to at least:
I) the first value associated with likelihood of epileptiform activity derived from one or more measurements of the patient's brain activity;
II) a second value associated with likelihood of epileptiform activity derived from one or more further measurements of the patient's brain activity; wherein the measurements for the second value are taken after the measurements taken for the first value.

10. The system as claimed in claim 9 wherein the system is configured to:
I) generate a first version of the model by fitting the output of the model to the first value, associated with likelihood of epileptiform activity derived from one or more measurements of the patient's brain activity;
II) updating the model by generating a second version of the model by fitting the output of the model to the first and second values associated with likelihood of epileptiform activity derived from one or more measurements of the patient's brain activity.

11. The system as claimed in claim 1 wherein the model comprises a set of two coupled stochastic differential equations for each considered brain region.

12. The system as claimed in claim 1 wherein the model is based off a bifurcation structure describing the transition between background brain states and seizure-like brain states.

13. A method for estimating the change over time of a likelihood of epileptiform activity of a patient; the method using a memory storing a model for estimating the likelihood of epileptiform activity of the patient; the model being configured to use data representing coupled brain activity for a plurality of different brain regions of the patient; the coupled brain activity associated with a brain network;
the method using a processor for:
I) determining one or more parameters for the model using one or more measurements of at least one physiological factor of the patient;
II) fitting the model, using the one or more parameters, to at least a first value associated with likelihood of epileptiform activity derived from one or more measurements of the patient's brain activity; the one or more measurements of the patient's brain activity for determining the data representing coupled brain activity in the model;
III) estimating the change over time of the likelihood of future epileptiform activity from the fitted model.

14-20. (canceled)

21. A method as claimed in claim 13 wherein the measurements comprise data associated with at least one physiological factor comprising:
a time-varying function associated with the physiological factor; and,
a weighting;
the processor configured to fit the model by varying the weighting and/or the time varying function.

22. A system as claimed in claim 13 wherein the measurements comprise data associated with one or more physiological factors comprises measurements of those physiological factors from the patient.

23. A system as claimed in claim 22 wherein the data associated with the physiological factor comprises any one of the following types:
IV) patient sleep data;
V) patient stress data;
VI) patient blood glucose data.

24. The system as claimed in claim 13 wherein the model describes:
III) the dynamic activity of each of the said brain regions with respect to time;
IV) the excitability within each of the brain regions with respect to time.

25. The system as claimed in claim 13 wherein the data representing coupled brain activity comprises data from a brain network model describing functional connections between the different brain regions.

26. The system as claimed in claim 13 wherein the first value associated with likelihood of epileptiform activity derived from one or more measurements of the patient's brain activity comprises data derived from EEG measurements of the patient's brain.

27. The system as claimed in claim 13 wherein:
the processor is configured to fit the model to at least:
III) the first value associated with likelihood of epileptiform activity derived from one or more measurements of the patient's brain activity;
IV) a second value associated with likelihood of epileptiform activity derived from one or more further measurements of the patient's brain activity;
wherein the measurements for the second value are taken after the measurements taken for the first value.
